(19) United States
(12) Patent Application Publication
(10) Pub. No.: US 2022/0118436 A1
(43) Pub. Date: Apr. 21, 2022

(54) CATALYST AND METHOD RELATED THERETO FOR THE SYNTHESIS OF HYDROCARBONS FROM SYNGAS

(71) Applicant: SABIC GLOBAL TECHNOLOGIES B.V., Bergen op Zoom (NL)

(72) Inventors: Jayen Barochia, Riyadh (SA); Khalid Karim, Riyadh (SA)

(21) Appl. No.: 17/422,848

(22) PCT Filed: Feb. 25, 2020
(86) PCT No.: PCT/IB2020/051599

§ 371 Date: Jul. 14, 2021

Related U.S. Application Data
(60) Provisional application No. 62/810,590, filed on Feb. 26, 2019.

(51) Int. Cl. B01J 37/30; B01J 23/75; B01J 37/08; B01J 37/04; B01J 6/00; C07C 1/04

(52) U.S. Cl. CPC B01J 37/30; B01J 23/75; B01J 37/08; C07C 2523/75; B01J 6/001; C07C 1/043; C07C 1/0445; B01J 37/04

(57) ABSTRACT

The present disclosures and inventions relate to a catalyst and methods for making same, which are useful in Fischer-Tropsch reactions.

### CROSS-REFERENCE TO RELATED APPLICATIONS

This application claims the benefit of U.S. Application No. 62/810,590, filed on Feb. 26, 2019, which is incorporated herein by reference in its entirety.

### FIELD OF THE INVENTION

The catalyst, composition, and method disclosed herein relate to catalysts for the conversion of hydrogen/carbon monoxide mixtures (syngas) to hydrocarbons.

### BACKGROUND

Syngas (mixtures of H₂ and CO) can be readily produced from either coal or methane (natural gas) by methods well known in the art and widely commercially practiced around the world. A number of well-known industrial processes use syngas for producing various hydrocarbons and oxygenated organic chemicals.

The Fischer-Tropsch catalytic process for catalytically producing hydrocarbons from syngas was initially discovered and developed in the 1920's, and was used in South Africa for many years to produce gasoline range hydrocarbons as automotive fuels. The catalysts typically comprised iron or cobalt supported on alumina or titania, and promoters, like rhenium, zirconium, manganese, and the like, were sometimes used with cobalt catalysts to improve various aspects of catalytic performance. The products were typically gasoline-range hydrocarbon liquids having six or more carbon atoms, along with heavier hydrocarbon products.

Today lower molecular weight hydrocarbons are desired and can be obtained from syngas via the Fischer-Tropsch catalytic process. Challenges exist to efficiently produce C2+ hydrocarbons at high yields without producing an excess of unwanted side products.

Accordingly, there remains a long-term market need for new and improved catalysts and methods related thereto for producing increased amounts of hydrocarbons, such as C2+ hydrocarbons, from syngas. Catalysts and methods useful for the production of hydrocarbons, such as C2+ hydrocarbons, from syngas are described herein.

### SUMMARY OF THE INVENTION

Disclosed herein is an ammonium modified Co based catalyst comprising Co, wherein the ammonium modified Co based catalyst has an ammonium modified surface

Also disclosed herein is a method comprising the step of:
a) contacting an alkali metal modified Co based catalyst with ammonium ions, thereby causing an exchange of alkali metal ions for ammonium ions on the alkali metal modified Co based catalyst, thereby producing an ammonium modified Co based catalyst.

Also disclosed herein is a method of producing C2+ hydrocarbons comprising contacting syngas with the ammonium modified CO based catalyst disclosed herein, thereby producing C2+ hydrocarbons.

Also disclosed herein is a composition comprising: a) as alkali metal modified Co based catalyst; b) an ammonium source; and c) a solvent.

Additional advantages will be set forth in part in the description which follows, and in part will be obvious from the description, or can be learned by practice of the aspects described below. The advantages described below will be realized and attained by means of the chemical compositions, methods, and combinations thereof particularly pointed out in the appended claims. It is to be understood that both the foregoing general description and the following detailed description are exemplary and explanatory only and are not restrictive.

### DETAILED DESCRIPTION OF THE FIGURES

These and other features of the preferred embodiments of the invention will become more apparent in the detailed description in which reference is made to the appended drawing.

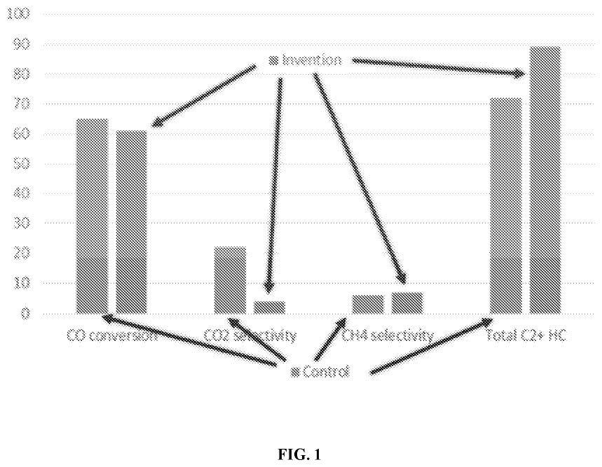

FIG. 1 shows data related to the conversion of syngas to hydrocarbons by a catalyst disclosed herein.

### DETAILED DESCRIPTION

Disclosed herein are materials, compounds, catalysts, compositions, and components that can be used for, can be used in conjunction with, can be used in preparation for, or are products of the disclosed method and compositions. It is to be understood that when combinations, subsets, interactions, groups, etc. of these materials are disclosed that while specific reference of each various individual and collective combinations and permutation of these compounds cannot be explicitly disclosed, each is specifically contemplated and described herein. For example, if a catalyst component is disclosed and discussed, and a number of alternative solid state forms of that component are discussed, each and every combination and permutation of the catalyst component and the solid state forms that are possible are specifically contemplated unless specifically indicated to the contrary. This concept applies to all aspects of this disclosure including, but not limited to, steps in methods of making and using the disclosed compositions. Thus, if there are a variety of additional steps that can be performed, it is understood that each of these additional steps can be performed with any specific aspect or combination of aspects of the disclosed methods, and that each such combination is specifically contemplated and should be considered disclosed.

In this specification and in the claims which follow, reference will be made to a number of terms which shall be defined to have the following meanings:

As used herein, the terms “Co based catalyst having an ammonium modified surface” and an “ammonium modified Co based catalyst” are used interchangeably. The terms “Co based catalyst having an ammonium modified surface” and an “ammonium modified Co based catalyst” mean that a Co based catalyst has ammonium ions present on its surface. It is noted that the Co based catalyst can also be ammonium modified in bulk mass.

The term “alkali metal modified Co based catalyst” means that a Co based catalyst has alkali metal ions present on its surface. It is noted that the Co based catalyst can also be alkali metal modified in bulk mass.

It must be noted that, as used in the specification and the appended claims, the singular forms “a,” “an” and “the” include plural referents unless the context clearly dictates otherwise. Thus, for example, reference to “a support material” includes mixtures of support materials.

“Optional” or “optionally” means that the subsequently described event or circumstance can or cannot occur, and that the description includes instances where the event or circumstance occurs and instances where it does not.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The word “or” as used herein means any one member of a particular list and also includes any combination of members of that list.

Ranges can be expressed herein as from “ ” one particular value, and/or to“ ” another particular value. When such a range is expressed, another aspect includes from the one particular value and/or to the other particular value. Similarly, when values are expressed as approximations, by use of the antecedent “,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight of component Y, X and Y are present at a weight ratio of 2:5, and are present in such a ratio regardless of whether additional components are contained in the compound.

A weight percent of a component, unless specifically stated to the contrary, is based on the total weight of the formulation or composition in which the component is included.

1. Co Based Catalyst for Producing the Co Based Catalyst

There is ongoing research to further develop sustainable technology of converting syngas to olefins, particularly light olefins, such as C2-C6 or C2-C4 olefins. Improving the catalyst used in this process is an important aspect of this development. Many catalytic regimes ranging from Cu, Co, La, Mn, Fe, Ni, Cr, Zr, etc. have been in focus over the past years (E. Schwab, A. Weck, J. Steiner, K. Bay, Oil Gas Eur. Mag. 1, 44-47 (2010); C. López, A. Corma, ChemCatChem 4, 751-752 (2012); M. E. Dry, “The Fischer-Tropsch process: 1950-2000” Catalysis Today, vol. 71, pp. 227-241, January 2002). Cobalt based catalysts are of particular interest as they show efficient activity at low temperatures i.e. high conversions and long-term stability as compared to other catalyst regimes (F. Diehl, and A. Y. Khodakov, “Promotion of Cobalt Fischer-Tropsch Catalysts with Noble Metals: a Review,” Oil Gas Sci. Technol.-Rev. IFP vol. 64, no. 1, pp. 11-24, November 2008; Vannice, M. A. J. Catal. 1975, 37, 449). Different attempts have been made to further enhance and improve the efficiency and selectivity towards desired products to improve the cobalt based catalyst regime (James Aluha et al Industrial & Engineering Chemistry Research (2015) 54(43), 10661-10674; Gregory R. Johnson et al ACS Catalysis 2015 S (10), 5888-5903).

Disclosed herein is an ammonium modified Co based catalyst comprising Co, wherein the ammonium modified Co based catalyst has an ammonium modified surface for converting syngas to hydrocarbons (a Fischer-Tropsch reaction), for example, selectively converting syngas to C2+ hydrocarbons, such as, for example, C₂-C₆ hydrocarbons or C₂-C₄ hydrocarbons. The ammonium modified Co based catalyst Co based catalyst disclosed herein has an improved yield and selectivity for converting syngas to C2+ hydrocarbons, such as, for example, C₂-C₆ hydrocarbons or C₂-C₄ hydrocarbons, as compared to conventional alkali metal modified catalysts.

The “Co based catalyst having an ammonium modified surface” and the “ammonium modified Co based catalyst” are produced from an alkali metal modified Co based catalyst where alkali metal ions are exchanged for ammonium ions.

The ammonium modified Co based catalyst disclosed herein can be prepared by the method disclosed herein.

The Fischer-Tropsch catalytic process for producing hydrocarbons from syngas is known in the art. Several reactions can take place in a Fischer-Tropsch process, such as, a Fischer-Tropsch (FT) reaction, a water gas shift reaction, and a hydrogen methanation, as shown in Scheme 1.

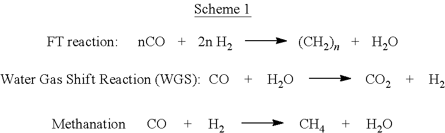

The ammonium modified Co based catalyst disclosed herein has a low water gas shift reaction activity as compared to a conventional catalyst, such as an alkali metal modified Co based catalyst. A low water gas shift reaction activity is desired as it provides a source of H₂ and CO₂ at the expense of CO and H₂O. Thus, during a Fischer-Tropsch process unwanted CO₂ is produced by the water gas shift reaction. The ammonium modified Co based catalyst disclosed herein has a low water gas shift activity as compared to a conventional alkali metal modified catalyst, thereby producing a low amount of CO₂ as shown herein. For example, the ammonium modified Co based catalyst disclosed herein has a water gas shift reaction that produces less than 8% or less than 5% CO₂ from the carbon monoxide (CO) feed. Accordingly, the ammonium modified Co based catalyst disclosed herein can have a CO₂ selectivity that is less than 8% or less than 5%.

The ammonium ion present on the surface of the ammonium modified Co based catalyst can be produced from an ammonium source selected from the group consisting of ammonia, ammonium carbonate, urea, ammonium chloride, ammonium hydroxide, ammonium tartrate, ammonium nitrate, and ammonium sulfate, or a combination thereof. In one example, the ammonium source comprises ammonia. In another example, the ammonium source comprises ammonium carbonate. In yet another example, the ammonium source comprises urea. In another example, the ammonium source comprises ammonium chloride. In another example, the ammonium source comprises ammonium hydroxide. In another example, the ammonium source comprises ammonium tartrate. In another example, the ammonium source comprises ammonium nitrate. In another example, the ammonium source comprises ammonium sulfate. The ammonium source provides the means for the ammonium ion that modifies the surface of a Co based catalyst.

In one aspect, the ammonium modified Co based catalyst comprises Co and Mn, wherein the ammonium modified Co based catalyst has an ammonium modified surface. For example, the ammonium modified Co based catalyst can comprise the formula CoMnₓO_{y}, wherein the molar ratio of x is from about 0.5 to about 1.5, and wherein the molar ratio of y is a number determined by the valence requirements of Co and Mn, wherein the ammonium modified Co based catalyst has an ammonium modified surface. In another example, the ammonium modified Co based catalyst can comprise the formula CoMnₓO_{y}, wherein the molar ratio of x is from about 0.8 to about 1.2, and wherein the molar ratio of y is a number determined by the valence requirements of Co and Mn, wherein the ammonium modified Co based catalyst has an ammonium modified surface. In yet another example, the ammonium modified Co based catalyst can comprise the formula CoMnₓSi_{z}O_{y}, wherein the molar ratio of x is from about 0.5 to about 1.5; wherein the molar ratio of z is from about 0.1 to about 1.0; and wherein the molar ratio of y is a number determined by the valence requirements of Co, Mn, and Si, wherein the Si is silica, wherein the ammonium modified Co based catalyst has an ammonium modified surface. In yet another example, the ammonium modified Co based catalyst can comprise the formula CoMnₓSi_{z}O_{y}, wherein the molar ratio of x is from about 0.8 to about 1.2; wherein the molar ratio of z is from about 0.1 to about 1.0; and wherein the molar ratio of y is a number determined by the valence requirements of Co, Mn, and Si, wherein the Si is silica, wherein the ammonium modified Co based catalyst has an ammonium modified surface.

In another aspect, the ammonium modified Co based catalyst comprises Co, Mn, and a promoter, wherein the ammonium modified Co based catalyst has an ammonium modified surface. Suitable promoters can be selected form the group consisting of lanthanum, chromium, vanadium, rhenium, phosphorous, ruthenium, boron, zinc, gallium, and a magnesium, or a combination thereof.

In one aspect, the ammonium modified Co based catalyst can be unsupported. In another aspect, the ammonium modified Co based catalyst can be supported. The support can comprise Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof. For example, the support can comprise Al₂O₃, SiO₂, TiO₂, CeO₂, AlPO₄, ZrO₂, MgO, or ThO₂, or a combination thereof.

In the Co based catalyst that comprises CoMnₓO_{y} or CoMnₓSi_{z}O_{y} disclosed herein can be non-stoichiometric solids, i.e. single phase solid materials whose composition cannot be represented by simple ratios of well-defined simple integers, because those solids probably contain solid state point defects (such as vacancies or interstitial atoms or ions) that can cause variations in the overall stoichiometry of the composition. Such phenomena are well known to those of ordinary skill in the arts related to solid inorganic materials, especially for transition metal oxides. Accordingly, for convenience and the purposes of this disclosure, the composition of the potentially non-stoichiometric catalytically active solids described herein will be quoted in ratios of moles of the other atoms as compared to the moles of cobalt and manganese ions or atoms in the same composition, whatever the absolute concentration of cobalt and manganese present in the composition. Accordingly, for purposes of this disclosure, the value of “x” and “z” are molar ratios relative to each other, regardless of the absolute concentration of cobalt and manganese in the catalyst. Thus, the subscript numbers represents molar ratios.

In the ammonium modified Co based catalyst that comprises the formula CoMnₓO_{y}, the molar ratio of manganese atoms to cobalt atoms, i.e. the value of “x” in the catalyst formula, can be from about 0.8 to about 1.2, from about 0.8 to about 1.1, from about 0.8 to about 1.0, from about 0.8 to about 0.9, from about 0.9 to about 1.2, from about 0.9 to about 1.1, from about 0.9 to about 1.0, from about 1.0 to about 1.2, or from about 1.0 to about 1.1. In one aspect, x can be about 1.0.

In the ammonium modified Co based catalyst that comprises the formula CoMnₓSi_{z}O_{y}, the molar ratio of manganese atoms to cobalt atoms, i.e. the value of “x” in the catalyst formula, can be from about 0.8 to about 1.2, from about 0.8 to about 1.1, from about 0.8 to about 1.0, from about 0.8 to about 0.9, from about 0.9 to about 1.2, from about 0.9 to about 1.1, from about 0.9 to about 1.0, from about 1.0 to about 1.2, or from about 1.0 to about 1.1. In one aspect, x can be about 1.0.

In the composition comprising the CoMnₓSi_{z}O_{y} catalyst, the molar ratio of Si atoms to cobalt atoms, i.e. the value of “z” in the catalyst formula, can be from about 0.1 to about 1.0, from about 0.3 to about 1.0, from about 0.5 to about 1.0, from about 0.7 to about 1.0, from about 0.1 to about 0.8, from about 0.3 to about 0.8, or from about 0.1 to about 0.5. In one aspect, y can be about 1.0 or about 0.5.

In one aspect, the molar ratio of x can be about 1.0 and the molar ratio of z can be from about 0.1 to about 1.0. In another aspect, the molar ratio of x can be from about 0.9 to about 1.1 and the molar ratio of z can be from about 0.1 to about 1.0. In yet another aspect, the molar ratio of x can be from about 0.9 to about 1.1 and the molar ratio of z can be from about 0.1 to about 0.8. In yet another aspect, the molar ratio of x can be from about 0.9 to about 1.1 and the molar ratio of z can be from about 0.5 to about 1.0.

In the ammonium modified Co based catalyst that comprises CoMnₓO_{y} or CoMnₓSi_{z}O_{y} disclosed herein, the molar ratio of oxygen atoms, i.e. the value of “y” in the catalyst formula, is a number determined by the valence requirements of Co and Mn, or and Co, Mn, and Si, depending of the formula. In one aspect, y is greater than 0 (zero). In another aspect, y can be 0 (zero). Even though a suitable catalyst composition of these inventions may be prepared or loaded into a reactor in the form of a mixed oxide (i.e. y is initially greater than 0), contact with hot syngas, either before or during the catalytic conversion of syngas to hydrocarbons begins, may result in the “in-situ” reduction of the catalyst composition and/or partial or complete removal of oxygen from the solid catalyst composition, with the result that y can be decreased to zero or zero. In one aspect, the value of y can be any whole integer or decimal fraction between 0 and 10. In some aspects of the catalyst described herein, y is greater than zero. In some aspects of the catalysts described herein, y can be from 1 to 5.

In one aspect, the ammonium modified Co based catalyst, such as CoMnₓO_{y} or CoMnₓSi_{z}O_{y} essentially consists of the ammonium modified Co based catalyst, such as CoMnₓO_{y} or CoMnₓSi_{z}O_{y} with or without a support material, wherein the CoMnₓO_{y} or CoMnₓSi_{z}O_{y} has an ammonium modified surface. In another aspect, the ammonium modified Co based catalyst, such as CoMnₓO_{y} or CoMnₓSi_{z}O_{y} essentially consists of the ammonium modified Co based catalyst, such as CoMnₓO_{y} or CoMnₓSi_{z}O_{y} with or without a support material, wherein the CoMnₓO_{y} or CoMnₓSi_{z}O_{y} has an ammonium modified surface.

2. Methods for Preparing the Catalyst

Also disclosed herein is a method of preparing an ammonium modified Co based catalyst, such as a ammonium modified Co based catalyst disclosed herein.

Accordingly, disclosed herein is a method comprising the step of:
a) contacting an alkali metal modified Co based catalyst with ammonium ions, thereby causing an exchange of alkali metal ions for ammonium ions on the alkali metal modified Co based catalyst, thereby producing an ammonium modified Co based catalyst.

In one aspect, the method further comprises drying the ammonium modified Co based catalyst. For example, the drying step can be performed at a temperature from 50° C. to 150° C. for a period of time, such as for at least 1 hr, 5 hrs, 12 hrs, or 24 hrs.

In one aspect, the method further comprises calcining the ammonium modified Co based catalyst. Calcining the ammonium modified Co based catalyst can performed in the presence of oxygen or air at high temperatures (such as for example exposing the ammonium modified Co based catalyst to a temperature of from, about 200° C. to about 800° C.), or similar heating under a dry inert gas such as nitrogen, can also be required in order to fully form the catalyst compositions. For example, calcining can result in the conversion of a physical mixture of components to form the catalyst phase, via various chemical reactions, such as for example the introduction of oxygen atoms or ions into the composition. In one aspect, the method further comprises calcining the ammonium modified Co based catalyst at a temperature from about 400° C. to about 600° C.

In one aspect, the method further comprises producing the alkali metal modified Co based catalyst comprising the steps of:
i) mixing a Co salt and a precipitating agent comprising an alkali metal in a solution, thereby producing an alkali metal modified Co based catalyst precursor; and
ii) drying and/or calcining the alkali metal modified Co based catalyst precursor, thereby producing the alkali metal modified Co based catalyst.

During the preparation of the alkali metal modified Co based catalyst, alkali metal ions from the precipitating agent adheres to the surface of the Co based catalyst precursor, thereby making it an alkali metal modified Co based catalyst precursor. The alkali metal modified Co based catalyst precursor is in turn dried and/or calcimined to produce the alkali metal modified Co based catalyst. The presence of the alkali metal ions on the surface of the alkali metal modified Co based catalyst promotes the activity of the unwanted water gas shift reaction in a Fischer-Tropsch process, thereby increasing the selectivity for the production of CO₂ in the Fischer-Tropsch process.

Contacting the alkali metal modified Co based catalyst with ammonium ions triggers an exchange of at least a portion of the alkali metal ions for an at least a portion of the ammonium ions to produce an ammonium modified Co based catalyst. The ammonium modified Co based catalyst does not promote the activity of the unwanted water gas shift reaction in the Fischer-Tropsch process as much as the alkali metal modified Co based catalyst. Therefore, the ammonium modified Co based catalyst has a decreased selectivity for the production of CO₂ in the Fischer-Tropsch process, as compared to a conventional alkali metal modified Co based catalyst. As described herein, the produced ammonium modified Co based catalyst can have a CO₂ selectivity that is less than 8% or less than 5%.

In one aspect, the precipitating agent comprising an alkali metal is a carbonate, bicarbonate, or hydroxide. In one aspect, the alkali metal is selected from the group consisting of sodium, potassium, lithium, cesium, rubidium, and francium. For example, the alkali metal can be selected from the group consisting of sodium, potassium, lithium, or cesium. In another example, the alkali metal is sodium. In yet another example, the alkali metal is potassium. In yet another example, the alkali metal is lithium. In yet another example, the alkali metal is cesium. In yet another example, the alkali metal is rubidium. In yet another example, the alkali metal is francium.

In one aspect, the precipitating agent comprising an alkali metal is selected from the group consisting of sodium carbonate, sodium bicarbonate, sodium hydroxide, potassium carbonate, potassium bicarbonate, potassium hydroxide, lithium carbonate, lithium bicarbonate, lithium hydroxide, cesium carbonate, cesium bicarbonate, or cesium hydroxide. For example, the precipitating agent comprising an alkali metal can be sodium carbonate. For example, the precipitating agent comprising an alkali metal can be sodium bicarbonate. For example, the precipitating agent comprising an alkali metal can be sodium hydroxide. For example, the precipitating agent comprising an alkali metal can be potassium carbonate. For example, the precipitating agent comprising an alkali metal can be potassium bicarbonate. For example, the precipitating agent comprising an alkali metal can be lithium carbonate. For example, the precipitating agent comprising an alkali metal can be lithium bicarbonate. For example, the precipitating agent comprising an alkali metal can be lithium hydroxide. For example, the precipitating agent comprising an alkali metal can be cesium carbonate. For example, the precipitating agent comprising an alkali metal can be cesium bicarbonate. For example, the precipitating agent comprising an alkali metal can be cesium hydroxide.

The concentration of the precipitating agent comprising an alkali metal can be varied in the method. In one aspect, the precipitating agent comprising the alkali metal can be used to alter the pH of the aqueous solution. For example, the mixing step of the method can comprise adding the precipitating agent comprising the alkali metal to the solution to adjust the pH of the solution to from about 6.5 to about 8.5, such as for example, to adjust the pH of the solution to from about 7.0 to about 7.5.

The step(s) of the methods for preparing the alkali modified Co based precursor catalyst described herein relates to providing a solution Co (cobalt atoms or ions (salts)), and optionally Mn (cobalt atoms or ions (salts)). Many suitable compounds comprising Co that are soluble in suitable solvents can be suitable and are known to those of ordinary skill in the art. In one aspect, water or low molecular weight alcohols, or mixtures thereof can be suitable solvents for this step. Any cobalt (II) or (III) salt that is soluble in an aqueous solution, such as water, can be used, and the use of cobalt (II) nitrate, cobalt tris(acetylacetonate), cobalt bis(acetylacetonate), cobalt (II) chloride, cobalt (II) bromide, cobalt (II) iodide, cobalt (II) acetate, cobalt (II) sulfate, and cobalt (II) diacetate, or a combination thereof are a specific examples of a suitable Co compound that can be dissolved to provide a suitable solution comprising Co. Any manganese (II) or (III) salt that is soluble in an aqueous solution, such as water, can be used, and the use of manganese (II) nitrate or manganese (II) acetate are a specific examples of suitable Mn compounds that can be dissolved to provide a suitable solution comprising Mn.

In one aspect, the solution comprises from about 0.1 mole % to about 2.0 mole %, such as for example, from about 0.5 mole % to about 1.5 mole %, of the cobalt salt prior to the formation of the alkali metal modified catalyst. In another aspect, the solution comprises from about 0.1 mole % to about 2.0 mole %, such as for example, from about 0.5 mole % to about 1.5 mole %, of the manganese salt prior to the formation of the alkali metal modified catalyst.

In one aspect, the solution comprising an aqueous or polar solvent, the aqueous or polar solvent is selected from the group consisting of water and glycol, or a combination thereof. In one aspect, the solution comprising an aqueous or polar solvent is water. In another aspect, the solution comprises water and glycol, wherein the glycol is selected from the group consisting of ethylene glycol, propylene glycol, dipropylene glycol, tripropylene glycol, and butylene glycol, or a combination thereof.

Accordingly, also disclosed herein is a composition comprising: a) an alkali metal modified Co based catalyst; b) an ammonium source; and c) a solvent.

In one aspect, the temperature of the solution is from about 20° C. to about 40° C. during the mixing step. In another aspect, the temperature of the solution is from about 25° C. to about 35° C. during the mixing step.

In one aspect, the method further comprises drying the alkali metal modified Co based catalyst precursor.

In one aspect of the methods for making the alkali metal modified Co based catalyst, the method further comprises calcining the alkali metal modified Co based catalyst precursor in the presence of oxygen or air at high temperatures (such as for example exposing the catalyst composition to a temperature of from, about 200° C. to about 800° C.), or similar heating under a dry inert gas such as nitrogen, can also be required in order to fully form the catalyst compositions. For example, calcining can result in the conversion of a physical mixture of components to form the catalyst phase, via various chemical reactions, such as for example the introduction of oxygen atoms or ions into the composition. In one aspect, the method further comprises calcining the alkali metal modified Co based catalyst precursor catalyst at a temperature from about 400° C. to about 600° C.

It is also to be understood that in some aspects of the compositions and methods described herein, once a catalyst has been formed by the methods described above, and the formed catalyst is loaded into reactors and contacted with syngas at reaction temperatures for significant periods of time, some physical and chemical changes can occur in the catalyst, either quickly or over time as the catalytic reactions with syngas are carried out. For example, contact of the metal oxide catalysts described herein with syngas at high temperatures can cause partial or complete “in-situ” reduction of the metal oxides, and such reduction processes can cause removal of oxygen atoms from the solid catalyst lattices, and/or cause reduction of some or all of the metal cations present in the catalyst to lower oxidation states, including reduction to metallic oxidation states of zero, thereby producing finely divided and/or dispersed metals on the catalyst supports. Such reduced forms of the catalysts of the invention are within the scope of the described compositions and methods.

The possible components and ranges of components for such compositions have already been described above, and can be applied in connection with describing and claiming methods for preparing such compositions.

In one aspect, the alkali metal modified Co based catalyst formed by the method disclosed herein can also be mixed with or dispersed on a support material. In one aspect, the alkali metal modified Co based catalyst is sprayed onto the support material. Suitable support materials include Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof.

In one aspect, the ammonium modified Co based catalyst formed by the method disclosed herein can also be mixed with or dispersed on a support material. In one aspect, the ammonium modified Co based catalyst is sprayed onto the support material. Suitable support materials include Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof.

In view of the general descriptions of the preparations of the catalyst compositions and variations thereof that are part of these inventions described above, herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.

3. Methods for Producing Hydrocarbons from Syngas

Described above is an ammonium modified Co based catalyst comprising Co, wherein the ammonium modified Co based catalyst has an ammonium modified surface and methods for making such a catalyst. The ammonium modified Co based catalyst is useful for converting mixtures of carbon monoxide and hydrogen (syngas) to hydrocarbons. The catalyst has unexpectedly low selectivity for converting syngas to CO₂, and a unexpectedly high yield for the production of C2+ hydrocarbons, such as to low molecular weight hydrocarbons such as C₂-C₆ hydrocarbons, such as, C₂-C₄ hydrocarbons from syngas.

Also disclosed herein is a method of producing C2+ hydrocarbons comprising contacting syngas with the ammonium modified CO based catalyst disclosed herein, thereby producing C2+ hydrocarbons, such as C₂-C₆ hydrocarbons, such as, C₂-C₄ hydrocarbons.

The ammonium modified CO based catalyst disclosed herein is reduced when present in conditions associated with process of producing C2+ hydrocarbons by contacting the catalyst composition with syngas. Such ammonium modified CO based catalyst is and can be referred to herein as a “reduced form of an ammonium modified CO based catalyst.” A reduction of the catalyst compositions under such conditions is known to those skilled in the art.

In these methods, mixtures of carbon monoxide and hydrogen (syngas) are contacted with suitable catalysts (whose composition, characteristics, and preparation have been already described above and in the Examples below) in suitable reactors and at suitable temperatures and pressures, for a contact time and/or at a suitable space velocity needed in order to convert at least some of the syngas to hydrocarbons. Unexpectedly as compared to methods in the prior art, the methods of the present inventions can have a low selectivity for the production of CO₂, and an unexpectedly high yield of production of C2+ hydrocarbons, which are valuable feedstocks for subsequent cracking processes at refineries for producing downstream products, such as low molecular weight olefins. C2+ hydrocarbons can be C₂-C₂ hydrocarbons, C₂-C₈ hydrocarbons, C₂-C₆ hydrocarbons, C₂-C₄ hydrocarbons or C₂-C₃ hydrocarbons.

Methods for producing syngas from natural gas, coal, or waste streams or biomass, at almost any desired ratio of hydrogen to carbon monoxide are well known to those of ordinary skill in the art. A large range of ratios of hydrogen to carbon monoxide can be suitable for the practice of the current invention, but since high conversion of carbon monoxide to hydrocarbons is desired, syngas mixtures comprising at least equimolar ratios of hydrogen to carbon monoxide or higher are typically employed, i.e. from 3:1 H₂/CO to 1:1 H₂/CO. In some aspects, the ratios of hydrogen to carbon monoxide employed are from 2:1 H₂/CO to 1:1 H₂/CO. Optionally, inert or reactive carrier gases, such as N₂, CO₂, methane, ethane, propane, and the like can be contained in and/or mixed with the syngas.

The syngas is typically forced to flow through reactors comprising the solid catalysts, wherein the reactors are designed to retain the catalyst against the vapor phase flow of syngas, at temperatures sufficient to maintain most of the hydrocarbon products of the catalytic reactions in the vapor phase at the selected operating pressures. The catalyst particles can be packed into a fixed bed, or dispersed in a fluidized bed, or in other suitable arrangements known to those of ordinary skill in the art.

In one aspect, the syngas is contacted with the catalyst compositions at a temperature of at least 200° C., or at least 300° C., and at a temperature below 400° C. or from a temperature of 200° C. to 350° C., or from a temperature of 230° C. to 270° C.

In one aspect, the syngas is contacted with the catalyst compositions at a pressure of at least 3 bar, 5 bar, or at least, 10 bar, or at least 15 bar, or at least 25 bar, or at least 50 bar, or at least 75 bar, and less than 200 bar, or less than 100 bar. In many aspects of the methods of the reaction, the syngas is contacted with the catalyst compositions at a pressure from 5 bar to 100 bar. In many aspects of the methods of the reaction, the syngas is contacted with the catalyst compositions at a pressure from about 3 bar to about 15 bar.

In one aspect, the syngas is contacted with the ammonium modified CO based catalyst to produce low amounts of CO₂. In one aspect, the method disclosed herein has a CO₂ selectivity of less than 8%. For example, the method disclosed herein can have a CO₂ selectivity of less than 7%. In another example, the method disclosed herein can have a CO₂ selectivity of less than 6%. In yet another example, the method disclosed herein can have a CO₂ selectivity of less than 5%. In yet another example, the method disclosed herein can have a CO₂ selectivity of less than 4%. In yet another example, the method disclosed herein can have a CO₂ selectivity of less than 3%. In yet another example, the method disclosed herein can have a CO₂ selectivity of less than 2%.

In one aspect, the disclosed herein has an unexpectedly highly selective for the production of C2+ hydrocarbons. Typical C2+ hydrocarbons, detected in the product include saturated hydrocarbons such as methane, ethane, propanes, butanes, and pentanes, and unsaturated hydrocarbons such as ethylene, propylene, butenes, and pentenes. In one aspect, the method has an unexpectedly higher selectivity for C₂-C₄ and C₂-C₃ hydrocarbons as compared to a reference alkali metal modified Co based catalyst not being contacted with ammonium ions.

In one aspect, the method has a total C2+ hydrocarbons selectivity of at least 80%. For example, the method can have a total C2+ hydrocarbons selectivity of at least 85%. In yet another example, the method has a total C2+ hydrocarbons selectivity of at least 90%.

In one aspect, the selectivity for production of C₂-C₄ hydrocarbons can be from about 10% to about 40%, from about 15% to about 30%, from about 20% to about 25%. In one aspect, the selectivity for production of C₂-C₃ hydrocarbons can be from about 10% to about 30%, from about 10% to about 20%, from about 15% to about 20%.

In view of the general descriptions of the catalyst compositions and variations thereof that are part of the inventions described above, herein below are described certain more particularly described aspects of methods for employing the catalysts for converting syngas to hydrocarbons. These particularly recited aspects should not however be interpreted to have any limiting effect on any different claims containing different or more general teachings, or that the “particular” aspects are somehow limited in some way other than the inherent meanings of the language and formulas literally used therein.

4. Aspects

In view of the described catalyst and catalyst compositions and methods and variations thereof, herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.

Aspect 1: A method comprising the step of: a) contacting an alkali metal modified Co based catalyst with ammonium ions, thereby causing an exchange of alkali metal ions for ammonium ions on the alkali metal modified Co based catalyst, thereby producing an ammonium modified Co based catalyst.

Aspect 2: The method of aspect 1, wherein the ammonium ions are produced from an ammonium source selected from the group consisting of ammonia, ammonium carbonate, urea, ammonium chloride, ammonium hydroxide, ammonium tartrate, ammonium nitrate, and ammonium sulfate, or a combination thereof.

Aspect 3: The method of aspects 1 or 2, wherein the alkali metal modified Co based catalyst comprises Co and Mn.

Aspect 4: The method of aspect 3, wherein the alkali metal modified Co based catalyst comprises Co, Mn, and a promoter.

Aspect 5: The method of any one of aspects 1-4, wherein the method further comprises drying the ammonium modified Co based catalyst.

Aspect 6: The method of any one of aspects 1-5, wherein the method further comprises calcining the ammonium modified Co based catalyst.

Aspect 7: The method of any one of aspects 1-6, wherein the method further comprises producing the alkali metal modified Co based catalyst comprising the steps of: i) mixing a Co salt and a precipitating agent comprising an alkali metal in a solution, thereby producing an alkali metal modified Co based catalyst precursor; and ii) drying and/or calcining the alkali metal modified Co based catalyst precursor, thereby producing the alkali metal modified Co based catalyst.

Aspect 8: The method of aspect 7, wherein step i) comprises mixing a Co salt, a Mn salt, and a precipitating agent comprising an alkali metal in a solution, thereby producing the alkali metal Co based catalyst precursor.

Aspect 9: The method of aspects 7 or 8, wherein the precipitating agent comprising an alkali metal is a carbonate, bicarbonate, or hydroxide.

Aspect 10: The method of any one of aspects 1-9, wherein the ammonium modified Co based catalyst comprises the formula CoMnₓO_{y}, wherein the molar ratio of x is from about 0.5 to about 1.5, and wherein the molar ratio of y is a number determined by the valence requirements of Co and Mn.

Aspect 11: An ammonium modified Co based catalyst produced by the method of any one of aspects 1-10.

Aspect 12: An ammonium modified Co based catalyst comprising Co, wherein the ammonium modified Co based catalyst has an ammonium modified surface.

Aspect 13: The ammonium modified Co based catalyst of aspect 12, wherein the ammonium modified Co based catalyst comprises Co and Mn, wherein the ammonium modified Co based catalyst has an ammonium modified surface.

Aspect 14: The ammonium modified Co based catalyst of aspect 12, wherein the ammonium modified Co based catalyst comprises Co, Mn, and a promoter, wherein the ammonium modified Co based catalyst has an ammonium modified surface.

Aspect 15: The ammonium modified Co based catalyst of aspect 12, wherein the ammonium modified Co based catalyst comprises the formula CoMnₓO_{y}, wherein the molar ratio of x is from about 0.5 to about 1.5, and wherein the molar ratio of y is a number determined by the valence requirements of Co and Mn, wherein the ammonium modified Co based catalyst has an ammonium modified surface.

Aspect 16: A method of producing C2+ hydrocarbons comprising contacting syngas with the ammonium modified CO based catalyst of any one of aspects 11-15, thereby producing C2+ hydrocarbons.

Aspect 17: The method of aspect 16, wherein the method has a CO₂ selectivity of less than 8%.

Aspect 18: The method of aspect 16, wherein the method has a CO₂ selectivity of less than 5%.

Aspect 19: The method of any one of aspects 16-18, wherein the method has a total C2+ hydrocarbons selectivity of at least 80%.

Aspect 20: A composition comprising a) an alkali metal modified Co based catalyst; b) an ammonium source; and c) a solvent.

### Examples

The following examples are put forth so as to provide those of ordinary skill in the art with a complete disclosure and description of how the compositions, catalysts, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric.

1. Control

Cobalt based control catalyst was synthesized by co-precipitation method using sodium carbonate as precipitating agent. 100 ml 1M solution of cobalt nitrate and 100 ml solution of manganese nitrate were premixed and heated to 60° C. 1M solution of sodium carbonate was heated to 60 C. Metal nitrate and sodium carbonate solutions were added simultaneously to 100 ml solution of 2 g of Aerosil 200 at 60° C. The pH was maintained to 7.5 throughout of addition. After addition complete, reaction mass was aged for 30 minute at 60 C. Precipitates were filtered, washed with hot water thoroughly to remove sodium ion followed by drying at 120° C. and calcination at 500° C. for 4 hours. The obtained catalyst is referred to as “control” in FIG. 1.

2. Invention

5% ammonia solution (100 ml) was heated to 60° C. 10 g of above prepared catalyst (example 1) was added to ammonia solution. Resulting slurry was stirred for 15 minutes followed by filtration and washing with hot water. Catalyst was dried at 120° C. and calcined at 500° C. for 2 h. The obtained catalyst is referred to as “invention” in FIG. 1. The term “invention” referred to in this example and in FIG. 1, is a non-limiting example of the method and ammonium modified Co based catalyst disclosed herein.

The data is FIG. 1 was obtained by testing the catalysts in in fixed bed reactor. 0.5 g of catalyst was fractioned in 60-80 mesh and loaded into the reactor with 1.5 g of inert material. The catalyst was activated by a reduction with 1:1 hydrogen and nitrogen mixture at 300° C. for 8 h. The catalyst activity was measured at 240° C., 5 bar, 2000 ml/g·h flow of feed. The syngas feed used for the reaction had a H₂/CO=2. 10% argon was used as internal standard. The outlet products were analyzed using online gas chromatography. Reported steady state data were collected after 100 h of TOS.

FIG. 1 shows that the catalyst that was exposed to the ammonia solution (“invention”) has a substantially lower CO₂ selectivity, as compared to the “control” catalyst that was not exposed to the ammonia solution. FIG. 1 also shows that the yield of C2+ hydrocarbons is substantially higher for the catalyst that was exposed to the ammonia solution (“invention”), as compared to the “control” catalyst that was not exposed to the ammonia solution.

1. A method comprising the step of:
a) contacting an alkali metal modified Co based catalyst with ammonium ions, thereby causing an exchange of alkali metal ions for ammonium ions on the alkali metal modified Co based catalyst, thereby producing an ammonium modified Co based catalyst.

2. The method of claim 1, wherein the ammonium ions are produced from an ammonium source selected from the group consisting of ammonia, ammonium carbonate, urea, ammonium chloride, ammonium hydroxide, ammonium tartrate, ammonium nitrate, and ammonium sulfate, or a combination thereof.

3. The method of claim 1, wherein the alkali metal modified Co based catalyst comprises Co and Mn.

4. The method of claim 3, wherein the alkali metal modified Co based catalyst comprises Co, Mn, and a promoter.

5. The method claim 1, wherein the method further comprises drying the ammonium modified Co based catalyst.

6. The method claim 1, wherein the method further comprises calcining the ammonium modified Co based catalyst.

7. The method of claim 1, wherein the method further comprises producing the alkali metal modified Co based catalyst comprising the steps of:
i) mixing a Co salt and a precipitating agent comprising an alkali metal in a solution, thereby producing an alkali metal modified Co based catalyst precursor; and
ii) drying and/or calcining the alkali metal modified Co based catalyst precursor, thereby producing the alkali metal modified Co based catalyst.

8. The method of claim 7, wherein step i) comprises mixing a Co salt, a Mn salt, and a precipitating agent comprising an alkali metal in a solution, thereby producing the alkali metal Co based catalyst precursor.

9. The method of claim 7, wherein the precipitating agent comprising an alkali metal is a carbonate, bicarbonate, or hydroxide.

10. The method claim 1, wherein the ammonium modified Co based catalyst comprises the formula CoMnₓO_{y}, wherein the molar ratio of x is from about 0.5 to about 1.5, and wherein the molar ratio of y is a number determined by the valence requirements of Co and Mn.

11. An ammonium modified Co based catalyst produced by the method of claim 1.

12. An ammonium modified Co based catalyst comprising Co, wherein the ammonium modified Co based catalyst has an ammonium modified surface.

13. The ammonium modified Co based catalyst of claim 12, wherein the ammonium modified Co based catalyst comprises Co and Mn, wherein the ammonium modified Co based catalyst has an ammonium modified surface.

14. The ammonium modified Co based catalyst of claim 12, wherein the ammonium modified Co based catalyst comprises Co, Mn, and a promoter, wherein the ammonium modified Co based catalyst has an ammonium modified surface.

15. The ammonium modified Co based catalyst of claim 12, wherein the ammonium modified Co based catalyst comprises the formula CoMnₓO_{y}, wherein the molar ratio of x is from about 0.5 to about 1.5, and wherein the molar ratio of y is a number determined by the valence requirements of Co and Mn, wherein the ammonium modified Co based catalyst has an ammonium modified surface.

16. A method of producing C2+ hydrocarbons comprising contacting syngas with the ammonium modified CO based catalyst of claim 11, thereby producing C2+ hydrocarbons.

17. The method of claim 16, wherein the method has a CO₂ selectivity of less than 8%.

18. The method of claim 16, wherein the method has a CO₂ selectivity of less than 5%.

19. The method of claim 16, wherein the method has a total C2+ hydrocarbons selectivity of at least 80%.

20. A composition comprising
a) an alkali metal modified Co based catalyst;
b) an ammonium source; and
c) a solvent.
